(19) United States
(12) Patent Application Publication
(10) Pub. No.: US 2009/0228006 A1
(43) Pub. Date: Sep. 10, 2009

(54) Apparatus for Treating Malformative and Traumatic Conditions Affecting the Knee Area

(75) Inventor: Pietro Mussolin, Monteviale (Vicenza) (IT)

Correspondence Address: COLEMAN SUDOL SAPONE, P.C., 714 COLORADO AVENUE, BRIDGE PORT, CT, 06605-1601, US

(73) Assignee: MIKAI S.P.A., Genova (IT)

(21) Appl. No.: 11/991,445

(22) PCT Filed: Sep. 5, 2006
(86) PCT No.: PCT/EP2006/008620

§ 371 Date: Mar. 4, 2008

(30) Foreign Application Priority Data
Sep. 15, 2005 (IT) PD2005 A 000266

(51) Int. Cl. A61B 17/56

(52) U.S. Cl. 606/59

(57) ABSTRACT

An apparatus for treating malformative and traumatic conditions affecting the knee area, having an external fixation means for fixing bone portions together and first and second bone screws to be engaged respectively in the epiphyseal and diaphyseal region of the femur/tibia and to which the fixation means is to be coupled. The apparatus includes a guiding means, which is separate from the fixation means, for guiding the surgeon in the operations for identifying a plurality of drilling points on these regions, for producing engagement holes in these regions at the drilling points, and for inserting the bone screws in the engagement holes. The separate provision of the guiding means allows to simplify the structure of the fixation means with respect to existing fixation means.

The present invention relates to an apparatus for treating malformative and traumatic conditions affecting the knee area. In particular, the apparatus is used in the surgical/orthopedic correction of axial deviations affecting the knee, known as genu varus and genu valgus, but also for the reduction of fractures affecting the metaphyseal region of the tibia and of the femur.

The apparatuses for treating conditions that affect the knee currently used in surgical/orthopedic technology include an external fixation means, suitable to fix bone portions together, and bone screws, to be engaged respectively within the epiphyseal and diaphyseal region of the femur/tibia and to which the fixation means is to be rigidly coupled. The fixation means incorporates a guiding means that guides the surgeon during the operations for identifying the drilling points on these regions, for providing engagement holes in these regions at the drilling points and for inserting the bone screws in the engagement holes. With this type of apparatus, the surgical procedure for correcting axial deviations of the knee, for example with a distraction osteotomy on the upper epiphysis of the tibia, occurs according to the following steps.

The patient is placed in a position suitable for performing the indicated surgical procedure and the surgeon performs palpation of the region of the knee in order to identify the overall operating area.

The surgeon places the fixation means next to the leg and positions it so that the upper part is approximately superimposed on the upper epiphysis of the tibia and the lower part is approximately superimposed on the tibial diaphysis. Subsequently, the surgeon introduces guiding wires in a first guiding means and then in the leg. The guiding wires have the function of temporarily fixing the fixation means to the leg. Then by means of second guiding means, the surgeon identifies a pair of drilling points in the area of the upper epiphysis of the tibia. The surgeon produces an incision in the tissues of the leg, at said drilling points and provides the corresponding engagement holes and inserts a pair of first bone screws. The upper part of the fixation means is rigidly coupled to the first bone screws. By means of a third guiding means the surgeon identifies a pair of drilling points in the diaphyseal region of the tibia. The leg is incised at said drilling points, producing the corresponding engagement holes and engaging second bone screws. The lower part of the fixation means is rigidly coupled to the second bone screws. Distraction osteotomy is performed in the metaphyseal region and the fixation means are finally adjusted. In the postoperative course, the adjustment initially given to the fixation means is changed periodically in an appropriate manner so as to promote progressive correction of the axial deviation that affects the knee. At the end of the postoperative course, the fixation means are disengaged from the bone screws and the screws are removed.

The main drawback observed in the apparatuses of the prior art is that the structure of those apparatuses is rather complex and surgeons find those apparatuses to be excessively laborious to apply during surgery. For example, it is observed that the position of application of the bone screws on the upper epiphysis of the tibia is determined according to the situations and preferences of the surgeon performing the procedure. Moreover, problems in terms of alignment and planarity are observed between the bone screws inserted in the epiphyseal region and the screws inserted in the diaphyseal region, causing the operation for rigidly coupling the fixation means to the bone screws to be rather complicated and awkward. In order to try to obviate these problems and prevent a badly performed implantation from negatively affecting the fracture reduction or correction process, the fixation means has been provided with several joints in order to allow the surgeon to rigidly couple in the best possible way the fixation means to the bone screws. However, that solution has caused a further encumbering of the overall structure of the fixation means. Those aspects consequently cause the success of the surgical procedure, and of the complete recovery of the limb, to be linked fundamentally to the experience and manual skill of the surgeon.

The aim of the present invention is to provide an apparatus, for treating malformative and traumatic conditions affecting the knee area, that is capable of overcoming the drawbacks of the prior art apparatuses.

An object of the invention is to provide an apparatus capable of significantly reducing the complexity and the procedural laboriousness of the application of bone crews and of a fixation means in procedures for treating conditions affecting the knee area.

A further object of the invention is to provide an apparatus that allows to provide implants, for correcting malformations or for reducing fractures of the knee, that are capable of promoting complete recovery of the limb following surgery.

A further object of the invention is to provide an apparatus that is more economic than known commercially available apparatuses.

A further object of the invention is to provide an apparatus that can be manufactured with known equipment and instruments.

This aim and these and other objects, which will become better apparent hereinafter, are achieved by an apparatus for treating malformative and traumatic conditions affecting the knee area, comprising an external fixation means for fixing bone portions together, first and second bone screws to be engaged respectively in the epiphyseal and diaphyseal regions of the femur/tibia, said fixation means being adapted to be coupled to said screws, characterized in that it comprises a guiding means which is separate from said fixation means for guiding the surgeon in the operations for identifying a plurality of drilling points on said regions, for providing engagement holes in said regions at said drilling points, and for inserting said bone screws in said engagement holes.

According to a first feature of the invention, the apparatus includes an external fixation means for fixing together bone portions; first and second bone screws, to be engaged respectively in the epiphyseal and diaphyseal region of the femur/tibia and to which the fixation means is to be coupled; and a guiding means which is separate from the fixation means in order to guide the surgeon in the operations for identifying a plurality of drilling points on these regions, for providing engagement holes in these regions at the drilling points and for inserting the bone screws in the engagement holes. The separate provision of the guiding means allows to simplify the structure of the fixation means, so as to make the procedure for applying the bone screws and the fixation means less complex and laborious. The guiding means allow to identify the drilling points, to produce the holes and to insert therein the corresponding bone screws so precisely that the fixation means couples to the bone screws without performing adaptations on the fixation means. This fact allows to provide implants that are executed in an optimum manner and decisively facilitate complete limb recovery.

According to further features of the invention, the guiding means includes an elongated body to be arranged along the longitudinal axis of the femur/tibia with a first terminal portion and a second terminal portion to be superimposed respectively on the epiphyseal region and on the diaphyseal region of the femur/tibia, a pair of first guiding holes and a pair of second guiding holes for guiding the surgeon in the operations for identifying the drilling points, for providing the engagement holes and for inserting the bone screws respectively in the epiphyseal region and in the diaphyseal region of the femur/tibia. The guiding means thus provided allows to reduce the cost of the apparatus as a whole, with respect to prior art apparatuses, and can be manufactured with conventional machinery and equipment.

Further characteristics and advantages will become better apparent from the description of the embodiment of the invention illustrated by way of non-limiting example in the accompanying four drawings, wherein:

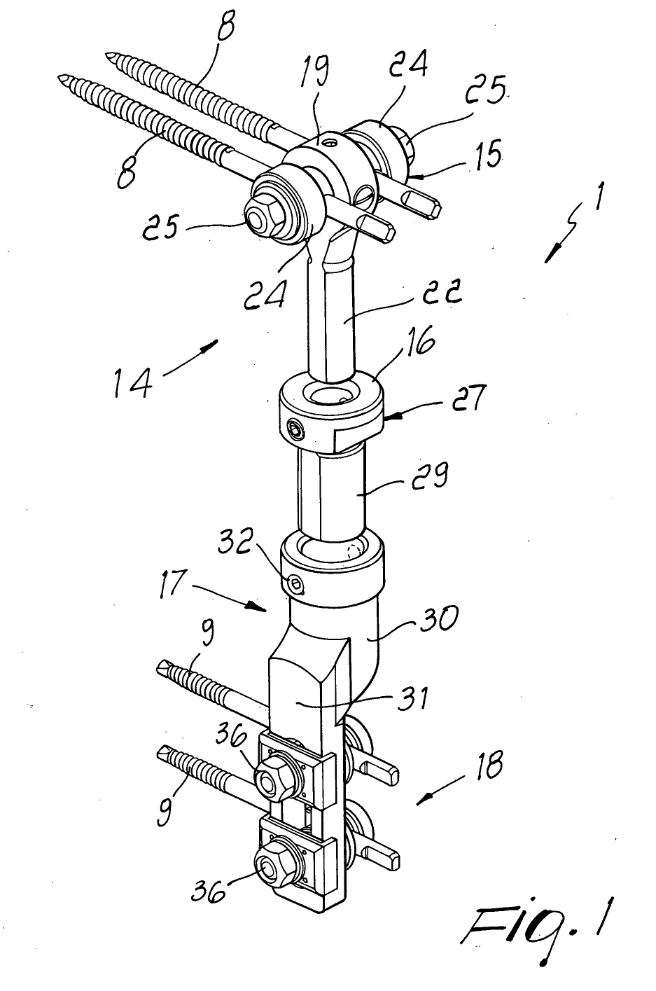

FIG. 1 is an exploded view of the fixation means and of the bone screws of the apparatus according to the invention;

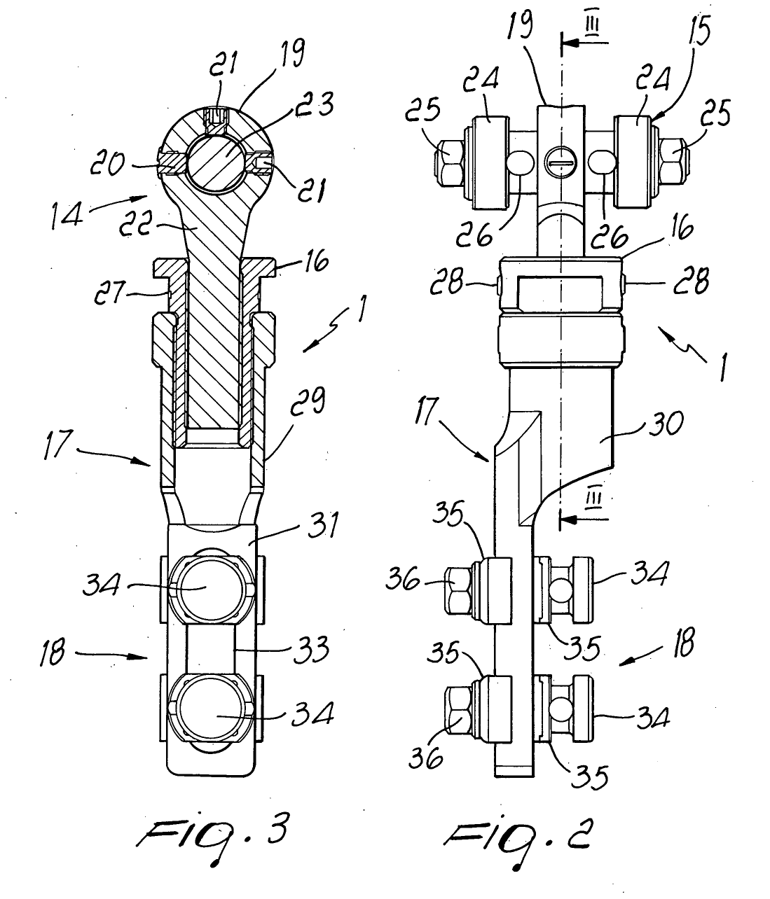

FIG. 2 is a rear view of the fixation means of FIG. 1;

FIG. 3 is a partially sectional side view, taken along the line III-III of FIG. 2, of the fixation means;

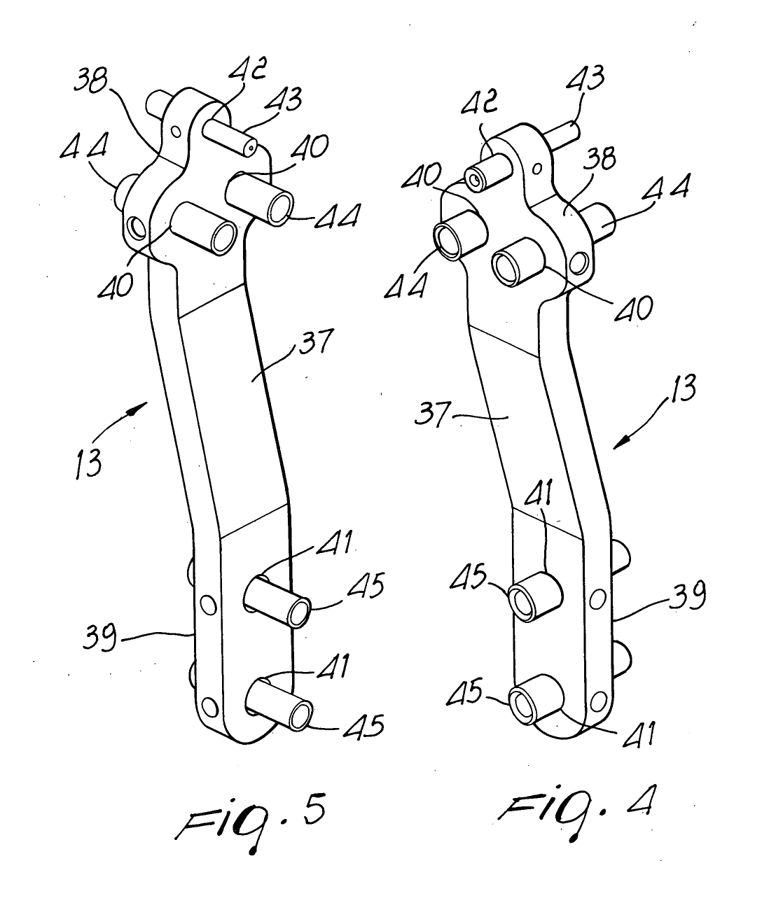

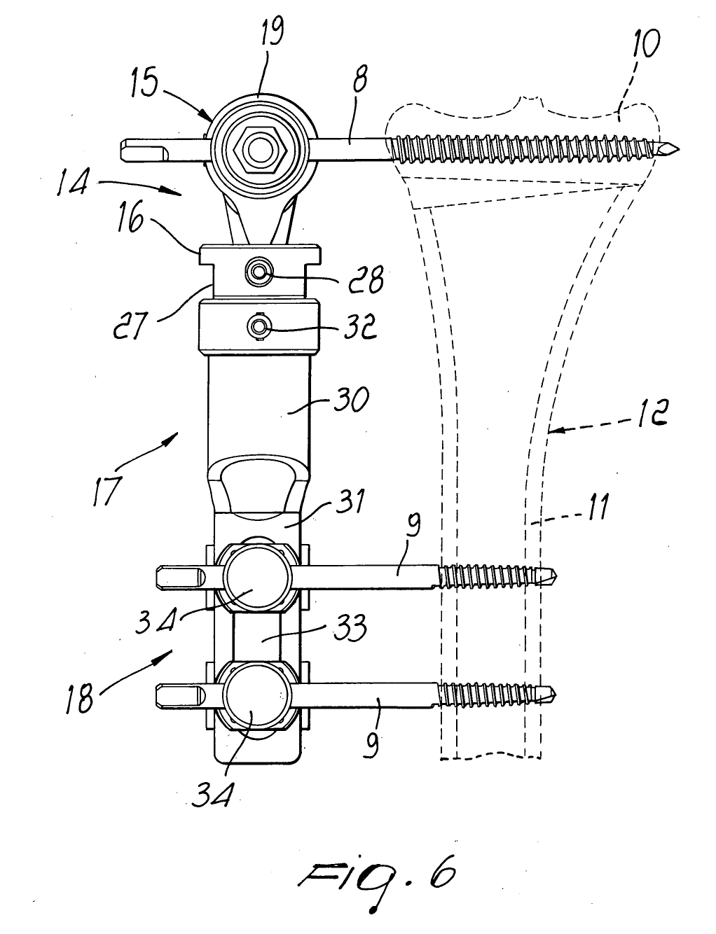

FIGS. 4 and 5 are perspective views, from opposite sides, of the guiding means of the apparatus according to the invention;

FIG. 6 is a side view of the fixation means and of the bone screws in the active condition on a tibia subjected to distraction osteotomy proximate to the upper epiphysis.

With reference to the figures of the accompanying drawings, the apparatus, according to the invention, comprises an external fixation means, generally designated by the reference numeral 1, for fixing bone portions together, and first and second bone screws 8 and 9, to be respectively engaged in the epiphyseal and diaphyseal regions 10, 11 of the tibia 12 and to which the fixation means 1 is to be rigidly coupled.

The apparatus also comprises a guiding means 13, which is separate from the fixation means 1, for guiding the surgeon in the operations for identifying a plurality of drilling points on these regions, for providing engagement holes in these regions at the drilling points and for the insertion in the engagement holes of the bone screws 8, 9.

The separate provision of the guiding means 13 allows to simplify the structure of the fixation means 1.

The fixation means 1 comprises a first supporting body 14, a first means 15 for rigidly coupling the first supporting body 14 to the first bone screws 8, an actuation body 16, which is connected to the first supporting body 14, a second supporting body 17, which is connected to the actuation body 16, and a second coupling means 18 for rigidly coupling the second supporting body 17 to the second bone screws 9.

The first supporting body 14 comprises an annular member 19, which partly accommodates the first coupling means 15. The first supporting body 14 also comprises three through holes provided so as to be angularly equidistant in a radial direction on the annular member 19, in order to respectively accommodate an extraction prevention screw 20, adapted to prevent the extraction of the first coupling means 15 from the annular member 19, and two first locking pins 21, for locking the first coupling means 15 and the first supporting body 14 together.

The first supporting body 14 also comprises an externally threaded wing 22, which protrudes on a plane from the peripheral region of the annular member 19 and is engaged by screw coupling in the actuation body 16.

The first coupling means 15 comprise a transverse pivot 23, which is inserted in the annular member 19 and engages the first bone screws 8.

Two spacers 24 are arranged proximate to the ends of the transverse pivot 23. Two lock nuts 25 lock the first bone screws 8 to the transverse pivot 23. The transverse pivot 23 comprises a central region for the engagement of the extraction preventing screw 20 and of the first locking pins 21.

Two through holes 26 are formed at the sides of the central region for the insertion of the first bone screws 8 engaging the lock nuts 25.

The actuation body 16 has a sleeve member, which is partly accommodated in the second supporting body 17. The actuation body 16 partly accommodates the first supporting body 14.

The sleeve member externally comprises a first portion 27, which forms an engagement seat for engaging an actuation tool (not shown). Two diametrically opposite through holes are provided at the seat in order to accommodate a pair of second locking pins 28 for locking the actuation body 16 and the first supporting body 14 together. A second portion 29 is provided with a first thread for coupling to the second supporting body 17.

The sleeve member also has a second internal thread for coupling to the first supporting body 14. The second thread is provided in the opposite direction with respect to the first thread so that, by turning the second actuation body 16 clockwise or counterclockwise, the first supporting body 14 is moved toward or away from the second supporting body 17. The second supporting body 17 has a first tubular portion 30, which partially accommodates the sleeve member. A second bar portion 31 supports two second coupling means 18. The tubular portion 30 has an internal thread for coupling to the first thread provided on the sleeve member. Two diametrically opposite through holes are provided in the collar of the tubular portion 30 in order to accommodate two third locking pins 32 for locking the second supporting body 17 and the actuation body 16 together. The bar portion 31 includes, in the central region, an elongated slot 33 for accommodating the pair of second coupling means 18. The coupling means include engagement pivots 34 for engaging the second bone screws 9. The second bone screws 9 are inserted in the slot 33. Through holes, for the insertion of the second bone screws 9, are provided proximate to a first end of the pivots 34. Plate members 35 are fitted on the pivots 34 at either end of the bar-like portion 30. Nuts 36, for locking the pivots 34, are engaged at the second end of the engagement pivots 34 in order to simultaneously lock the bone screws 9 to the second coupling means 18 and to lock the coupling means to the bar-like portion 30. The guiding means has an elongated body 37 to be arranged along the longitudinal axis of the tibia 12 with a first terminal portion 38 and a second terminal portion 39 to be superimposed respectively on the epiphyseal region 10 and on the diaphyseal region 11 of the tibia 12. Two of first guiding holes 40 and two second guiding holes 41 guide the surgeon in identifying the drilling points, for producing the holes for the engagement and insertion of the bone screws 8 and 9 respectively in the epiphyseal region 10 and in the diaphyseal region 11 of the tibia 12. The guiding means also includes a passage hole 42 for passing a guiding wire (not shown) inserted in the knee region. The hole is provided proximate to the end of the first terminal portion 38. The pair of first guiding holes 40 is formed in the first terminal portion 38 in a region adjacent to the passage hole 42 and is aligned transversely to the longitudinal axis of the body 37. The second guiding holes 41 are provided in the second terminal portion 39 and are longitudinally aligned. The first and second terminal portions 38 and 39 are arranged on offset planes. The passage hole 42 and the pairs of first and second guiding holes 40, 41 partially accommodate tubular bodies 43, 44, 45 in order to facilitate the guiding of the drilling bits and of the bone screws.

With particular reference to FIG. 6, the procedure for applying the apparatus is described hereafter with reference to the execution of a surgical procedure for distraction osteotomy affecting the upper part of the tibia. The steps of accommodating the patient and of palpation occur as described above. In a first step, the surgeon inserts one end of a guiding wire in the knee, at the level of the tibial plate, and inserts the other end of the same guiding wire in the tubular body 43 of the guiding means 13.

In a second step, the surgeon arranges the elongated body 37 in the position described above and presses it against the leg, so that the tubular bodies 44, 45 leave impressions on the skin of the leg. Those impressions identify the drilling holes. In a third step, the surgeon removes the elongated body 37 from the operating area and incides the tissues of the leg at the drilling points.

In a fourth step, the surgeon places the elongated body 37 again adjacent to the leg until it assumes the previous arrangement, and then, with one hand, keeps it pressed against the leg and with the other hand drills a first engagement hole in the region of the upper epiphysis 10 of the tibia 12, inserting the drilling bit of the drilling means in one of the tubular bodies 44.

In a fourth step, the surgeon keeps the elongated body 37 in the previous condition, inserts a bone screw 8 in the tubular body 44 and then inserts the bone screw 8 in the engagement hole that he has just provided. In a fifth step, the surgeon keeps the tubular body 37 in the preceding condition and produces the remaining engagement holes by inserting in succession the drilling bit in the remaining tubular bodies 44 and 45. Then the surgeon inserts the remaining bone screws 8, 9 in the remaining tubular bodies 44, 45 and inserts them in the engagement holes that he has just provided.

The first sequence of the procedure ends with the removal of the guiding means 13 from the operating area. In the second sequence, the surgeon performs distraction osteotomy proximate to the upper epiphysis 10 of the tibia 12. The surgeon applies the fixation means 7 to the bone screws 8, 9, coupling the first bone screws 8 to the first coupling means 15 and the second bone screws 9 to the second coupling means 18.

Then the surgeon performs final adjustment of the fixation means 7.

During the postoperative course, the adjustment initially given to the fixation means 7 is changed periodically and appropriately by means of the actuation body 16 in order to promote the progressive correction of the axial deviation that affects the knee.

In practice it has been found that the apparatus achieves the intended aim and objects, overcoming the drawbacks observed in the background art. The apparatus in fact allows to significantly reduce the procedural laboriousness and complexity of application of bone screws and fixation means in procedures for treating pathologies affecting the knee area. The apparatus is capable of promoting complete recovery of the limb after surgery. The apparatus is more economic than prior art commercially available apparatuses and can be produced with traditional instruments and equipment.

In practical execution, the materials used, the shapes, the dimensions and the constructive details may be different from the ones described above, but technically equivalent, within the scope of the appended claims.

This application claims the priority of Italian Patent Application No. PD2005A000266,filed on Sep. 15, 2005, the subject matter of which is incorporated herein by reference.

1. An apparatus for treating malformative and traumatic conditions affecting the knee area, comprising an external fixation means for fixing bone portions together, first and second bone screws to be engaged respectively in the epiphyseal and diaphyseal regions of the femur/tibia, said fixation means being adapted to be coupled to said screws, further comprising a guiding means which is separate from said fixation means for guiding the surgeon in the operations for identifying a plurality of drilling points on said regions, for providing engagement holes in said regions at said drilling points, and for inserting said bone screws in said engagement holes.

2. The apparatus according to claim 1, wherein said fixation means also comprises a first supporting body, a first coupling means for coupling said first supporting body to said first bone screws, an actuation body which is connected to said first supporting body, a second supporting body which is connected to said actuation body, and a second coupling means for coupling said second supporting body to said second bone screws.

3. The apparatus according to claim 2, wherein said first supporting body comprises an annular member which partly accommodates said first coupling means, at least three through holes provided so as to be angularly equidistant in a radial direction on said annular member in order to accommodate respectively an extraction preventing screw, to prevent the extraction of said first coupling means from said annular member, and two first locking pins for locking together said first coupling means and said first supporting body, and an externally threaded wing which protrudes on a plane from the peripheral region of said annular member and is engaged by a screw coupling in said actuation body.

4. The apparatus according to claim 2, wherein said first coupling means comprises a transverse pivot which is inserted in said annular member and engages said first bone screws, two spacers arranged proximate to the ends of said transverse pivot, and two lock nuts for locking said first bone screws to said transverse pivot which are engaged at the ends of said transverse pivot.

5. The apparatus according to claim 4, wherein said transverse pivot comprises a central region for the engagement of said extraction prevention screw and of said first locking pins, two through holes provided at the sides of said central region for the insertion of said first bone screws, and threaded terminal portions for coupling to said lock nuts.

6. The apparatus according to claim 2, wherein said actuation body comprises a sleeve member, which is partially accommodated within said second supporting body and in turn accommodates partially said first supporting body.

7. The apparatus according to claim 6, wherein said sleeve member externally comprises a first portion, which forms an engagement seat for engaging an actuation tool, two through holes provided at said seat in a diametrically opposite condition in order to accommodate two second locking pins for locking together said actuation body and said first supporting body, a second portion provided with a first thread for coupling to said second supporting body, and internally a second thread for coupling to said first supporting body, said second thread being provided in the opposite direction with respect to said first thread.

8. The apparatus according to claim 6, wherein said second supporting body comprises a tubular portion, which partially accommodates said sleeve member, and a bar portion, which supports said second coupling means.

9. The apparatus according to claim 8, wherein said tubular portion comprises an internal thread for coupling to said first thread, which is formed on said sleeve member, and two through holes provided in a diametrically opposite condition in the collar of said tubular portion in order to accommodate two third locking pins in order to lock together said second supporting body and said actuation body.

10. The apparatus according to claim 8, wherein said bar portion comprises, in the central region, an elongated slot for accommodating said second coupling means.

11. The apparatus according to claim 10, wherein said second coupling means comprises engagement pivots for engaging said second bone screws that are inserted in said slot, through holes for the insertion of said second bone screws, said through holes being provided proximate to first ends of said pivots, plate members fitted on said pivots at either end of said bar portion, nuts for locking said pins and engaged at the second end of said engagement pivots in order to simultaneously lock said bone screws to said coupling means and said coupling means to said bar portion.

12. The apparatus according to claim 1, wherein said guiding means comprises an elongated body to be arranged along the longitudinal axis of said femur/tibia with a first terminal portion and a second terminal portion to be superimposed respectively on said epiphyseal region and on said diaphyseal region of said femur/tibia, two first guiding holes and two second guiding holes for guiding said surgeon in said operations for identifying said drilling points, for producing said engagement holes and for inserting said bone screws respectively in said epiphyseal region and in said diaphyseal region of said femur/tibia.

13. The apparatus according to claim 12, wherein said guiding means comprises a passage hole for passing a guiding wire which is inserted in the region of said knee, said hole being provided proximate to the end of said first terminal portion.

14. The apparatus according to claim 13, wherein said first guiding holes are provided in said terminal portion in a region which is adjacent to said passage hole and are aligned transversely to the longitudinal axis of said body, said second guiding holes being provided in said second terminal portion and being aligned in a longitudinal direction.

15. The apparatus according to claim 12, wherein said first and second terminal portions lie on offset planes.

16. The apparatus according to claim 13, wherein said passage hole and said pairs of first and second guiding holes partially accommodate tubular bodies to facilitate the guiding of drilling bits and of said screws.
